SEQUENCE LISTING (41 sequences)

SEQ ID NO: 1 - protein, 9 aa, African swine fever virus
TVSAIELEY

SEQ ID NO: 2 - protein, 9 aa, African swine fever virus
KTRDFFILY

SEQ ID NO: 3 - protein, 9 aa, African swine fever virus
MMDFERVHY

SEQ ID NO: 4 - protein, 9 aa, African swine fever virus
KNLSIIWEY

SEQ ID NO: 5 - protein, 9 aa, African swine fever virus
KAIELYWVF

SEQ ID NO: 6 - protein, 9 aa, African swine fever virus
YLYEIEIEY

SEQ ID NO: 7 - protein, 9 aa, African swine fever virus
SKENLTPDE

SEQ ID NO: 8 - protein, 12 aa, African swine fever virus
NECTSSFETLFE

SEQ ID NO: 9 - protein, 12 aa, African swine fever virus
SALRHLDPKKLD

SEQ ID NO: 10 - protein, 9 aa, African swine fever virus
KYLDDNSLL

SEQ ID NO: 11 - protein, 6 aa, African swine fever virus
QQWVEV

SEQ ID NO: 12 - protein, 10 aa, African swine fever virus
HKPHQSKPIL

SEQ ID NO: 13 - protein, 15 aa, African swine fever virus
ESVYFAVETIHLKQQ

SEQ ID NO: 14 - protein, 15 aa, African swine fever virus
KIGFYSSKSTAHERE

SEQ ID NO: 15 - protein, 15 aa, African swine fever virus
TSLFTIAISNRDLQL

SEQ ID NO: 16 - protein, 9 aa, African swine fever virus
SKENLTPDE

SEQ ID NO: 17 - protein, 12 aa, African swine fever virus
NECTSSFETLFE

SEQ ID NO: 18 - protein, 6 aa, African swine fever virus
QQWVEV

SEQ ID NO: 19 - protein, 10 aa, African swine fever virus
SNIKNVNKSY

SEQ ID NO: 20 - protein, 15 aa, African swine fever virus
KFTVVTVKAKPARQG

SEQ ID NO: 21 - protein, 15 aa, African swine fever virus
KGNYYIKNTKAGSLI

SEQ ID NO: 22 - protein, 16 aa, African swine fever virus
GIAGRGIPLGNPHVKP

SEQ ID NO: 23 - protein, 12 aa, African swine fever virus
LDAVKMDKRNIK

SEQ ID NO: 24 - protein, 16 aa, African swine fever virus
AKLQDTKFKWKYTLDP

SEQ ID NO: 25 - protein, 11 aa, African swine fever virus
GRPSRRNIRFK

SEQ ID NO: 26 - protein, 5 aa, African swine fever virus
GPGPG

SEQ ID NO: 27 - protein, 5 aa, African swine fever virus
EAAAK

SEQ ID NO: 28 - protein, 5 aa, African swine fever virus
GGGGS

SEQ ID NO: 29 - protein, 420 aa, African swine fever virus
MSKLSSDELLDVFKEMTLLELSDFVKKFEETFEVTAAAPVSVAVAGAPAAGEAGEAAEEQSEFDVILESAGDKKIGVIKVVREIVSGLGLKEAKDLVDGVPKLLLEKVAKEAADDAKAKLEATGATVSVKEAAAKTVSAIELEYAAYKTRDFFILYAAYMMDFERVHYAAYKNLSIIWEYAAYKAIELYWVFAAYYLYEIEIEYGPGPGESVYFAVETIHLKQQGPGPGKIGFYSSKSTAHEREGPGPGTSLFTIAISNRDLQLGPGPGKFTVVTVKAKPARQGGPGPGKGNYYIKNTKAGSLIEAAAKSKENLTPDEKKNECTSSFETLFEKKQQWVEVKKSNIKNVNKSYKKGIAGRGIPLGNPHVKPKKLDAVKMDKRNIKKKAKLQDTKFKWKYTLDPKKGRPSRRNIRFKGGGGS

SEQ ID NO: 30 - RNA, 1266 nt, African swine fever virus
  source 1..1266: mol_type = mRNA
atgatgtctaagctgtcttctgatgagctgctggatgtgtttaaggagatgactctgctggagctgtctgattttgtgaagaagtttgaggagacttttgaggtgactgctgctgctcctgtgtctgtggctgtggctggtgctcctgctgctggtgaggctggtgaggctgctgaggagcagtctgagtttgatgtgattctggagtctgctggtgataagaagattggtgtgattaaggtggtgcgtgagattgtgtctggtctgggtctgaaggaggctaaggatctggtggatggtgtgcctaagctgctgctggagaaggtggctaaggaggctgctgatgatgctaaggctaagctggaggctactggtgctactgtgtctgtgaaggaggctgctgctaagactgtgtctgctattgagctggagtatgctgcttataagactcgtgatttttttattctgtatgctgcttatatgatggattttgagcgtgtgcattatgctgcttataagaacctgtctattatttgggagtatgctgcttataaggctattgagctgtattgggtgtttgctgcttattatctgtatgagattgagattgagtatggtcctggtcctggtgagtctgtgtattttgctgtggagactattcatctgaagcagcagggtcctggtcctggtaagattggtttttattcttctaagtctactgctcatgagcgtgagggtcctggtcctggtacttctctgtttactattgctatttctaaccgtgatctgcagctgggtcctggtcctggtaagtttactgtggtgactgtgaaggctaagcctgctcgtcagggtggtcctggtcctggtaagggtaactattatattaagaacactaaggctggttctctgattgaggctgctgctaagtctaaggagaacctgactcctgatgagaagaagaacgagtgtacttcttcttttgagactctgtttgagaagaagcagcagtgggtggaggtgaagaagtctaacattaagaacgtgaacaagtcttataagaagggtattgctggtcgtggtattcctctgggtaaccctcatgtgaagcctaagaagctggatgctgtgaagatggataagcgtaacattaagaagaaggctaagctgcaggatactaagtttaagtggaagtatactctggatcctaagaagggtcgtccttctcgtcgtaacattcgttttaagggtggtggtggttcttga

SEQ ID NO: 31 - protein, 8 aa, African swine fever virus
GAHRMDIR

SEQ ID NO: 32 - protein, 11 aa, African swine fever virus
DICLPRWGCLW

SEQ ID NO: 33 - protein, 17 aa, African swine fever virus
ADQVSDQTLNAVVHVFK

SEQ ID NO: 34 - protein, 17 aa, African swine fever virus
NQDKQLAALNDVGAKAA

SEQ ID NO: 35 - protein, 8 aa, African swine fever virus
FYWHCLDE

SEQ ID NO: 36 - protein, 18 aa, African swine fever virus
RLIEDICLPRWGCLWEDD

SEQ ID NO: 37 - protein, 8 aa, African swine fever virus
QYDDAVRR

SEQ ID NO: 38 - protein, 7 aa, African swine fever virus
TNLLSAQ

SEQ ID NO: 39 - protein, 7 aa, African swine fever virus
WQVDGRG

SEQ ID NO: 40 - protein, 7 aa, African swine fever virus
FNKYIEH

SEQ ID NO: 41 - protein, 7 aa, African swine fever virus
LGGHAEK

(19) United States
(12) Patent Application Publication
(10) Pub. No.: US 2026/0273039 A1
(43) Pub. Date: Sep. 17, 2026

(54) UNIVERSAL VACCINE FOR AFRICAN SWINE FEVER VIRUS (ASFV) INFECTIONS

(71) Applicant: DEI Biopharma, Kampala (UG)

(72) Inventor: Matthias Magoola, Kampala (UG)

(21) Appl. No.: 19/082,038

(22) Filed: Mar. 17, 2025

(51) Int. Cl. A61K 39/12; A61K 39/00; A61K 39/385; A61K 39/39

(52) U.S. Cl. CPC A61K 39/12; A61K 39/385; A61K 39/39; A61K 2039/53; A61K 2039/54; A61K 2039/542; A61K 2039/543; A61K 2039/552; A61K 2039/55555; A61K 2039/55561; A61K 2039/55566; A61K 2039/55577; A61K 2039/6031; A61K 2039/6081; A61K 2039/70

(57) ABSTRACT

The present invention relates to vaccine compositions for administration to domestic pigs (Sus scrofa domesticus), wild boars, warthogs, bush pigs, and giant forest hogs. In one embodiment, the present vaccine is designed to provide broad-spectrum immunity against all known genotypes (I-XXIV) and serotypes (1-8) of African Swine Fever Virus (ASFV) by incorporating highly conserved antigenic regions across multiple viral proteins. This multi-epitope vaccine construct is formulated to elicit both cellular (CD8⁺ and CD4⁺ T-cell) and humoral (antibody-mediated) immunity, ensuring cross-protection against diverse ASFV strains. By targeting conserved epitopes in structural (p72, p54, p30, CD2v) and non-structural proteins (MGF family, DNA replication enzymes, immune evasion factors), this vaccine aims to mitigate ASFV outbreaks globally, including regions affected by highly virulent Genotype II (Eastern Europe, Asia) and endemic strains in Africa. This universal antigen is optionally fused to a serum protein or a small synthetic peptide capable of binding to a serum protein in vivo to extend its half-life. In another embodiment, the invention, the antigen is produced by recombinant technology, or RNA technology to deliver the antigen in vivo or to produce it by a cell-free synthesis.

### SEQUENCE LISTING

The instant application contains a Sequence Listing, which has been submitted electronically in .xml format and is hereby incorporated by reference in its entirety. Said text file, created on 17 Mar. 2025, is named Universal African Swine Fever.xml and is 37000 byte in size.

### FIELD OF THE INVENTION

The present invention relates to vaccine compositions for administration to domestic pigs (Sus scrofa domesticus), wild boars, warthogs, bush pigs, and giant forest hogs. In one embodiment, the present vaccine is designed to provide broad-spectrum immunity against all known genotypes (I-XXIV) and serotypes (1-8) of African Swine Fever Virus (ASFV) by incorporating highly conserved antigenic regions across multiple viral proteins. This multi-epitope vaccine construct is formulated to elicit both cellular (CD8⁺ and CD4⁺ T-cell) and humoral (antibody-mediated) immunity, ensuring cross-protection against diverse ASFV strains. By targeting conserved epitopes in structural (p72, p54, p30, CD2v) and non-structural proteins (MGF family, DNA replication enzymes, immune evasion factors), this vaccine aims to mitigate ASFV outbreaks globally, including regions affected by highly virulent Genotype II (Eastern Europe, Asia) and endemic strains in Africa. This universal antigen is optionally fused to a serum protein or a small synthetic peptide capable of binding to a serum protein in vivo to extend its half-life. In another embodiment, the invention, the antigen is produced by recombinant technology, or RNA technology to deliver the antigen in vivo or to produce it by a cell-free synthesis.

### BACKGROUND OF THE INVENTION

The animal health industry is a key player in this challenge and strives to develop advanced solutions for a safe, secure, and sustainable food supply. Animal health products contribute to improving and maintaining the health and welfare of animals in terms of disease prevention, treatment, and control. One of the most important causes of animal diseases is infections by viruses. Some are contagious, can produce devastating economic losses, and impact public health; the African swine fever virus (ASFV) has an almost 100% mortality rate, making it one of the most serious diseases that cause productivity losses and economic impacts by affecting domestic pigs (Sus scrofa domesticus), wild boars, warthogs, bush pigs, and giant forest hogs.

African Swine Fever Virus (ASFV) has no direct utility but has significant implications in agriculture, science, and biosecurity. It poses a severe threat to the global pork industry, causing African Swine Fever (ASF) with up to 100% mortality in pigs. Without a commercial vaccine or treatment, outbreaks result in devastating economic losses, as seen in China's 2018-2019 crisis, where over 50% of the pig population was lost. ASFV research has advanced vaccine technologies, including mRNA, viral vectors, and protein-based approaches. It offers insights into viral immunity and host-pathogen interactions, particularly for large DNA viruses like poxviruses and herpesviruses. As one of the few cytoplasmic-replicating DNA viruses, ASFV provides a model for studying viral replication and immune evasion, contributing to antiviral drug development and gene therapy strategies.

Additionally, ASFV is a biosecurity concern due to its stability and high transmissibility, raising agroterrorism threats. Its persistence in wild boars and soft ticks complicates eradication efforts and impacts ecosystem balance. While ASFV has no beneficial use, it remains a high-priority pathogen for research, given its role in virology, vaccine development, and disease control.

African Swine Fever (ASF) has inflicted substantial economic losses on the global pork industry. In China, which previously accounted for half of the world's pork production, the ASF outbreak led to the culling of approximately 40% of its pig population, resulting in direct economic losses estimated at $141 billion by September 2019. The outbreak also caused a 30% reduction in pork production and a 40% increase in pork prices over a year.

In Vietnam, within the first five months of the ASF outbreak in 2019, about 20% of pigs died or were culled, leading to economic losses estimated between $880 million and $4.4 billion. Similarly, India reported losses of $37.32 million due to ASF-related swine mortality from June to April 2020.

The United States, although not currently affected, faces significant risks. A hypothetical ASF outbreak could lead to a loss of export markets, reducing the value of each carcass by approximately $10. This scenario could result in first-year losses of about $8 billion and the potential loss of 60,000 jobs if the disease is not controlled promptly.

In Europe, Italy's renowned pork industry, valued at €20 billion, has been severely impacted by ASF. Since the disease's detection in January 2022, nearly 120,000 pigs have been culled to contain its spread, leading to significant financial losses for farmers and affecting the production of products like Parma prosciutto.

Overall, ASF has disrupted global pork production, leading to decreased income for farmers, increased prices for consumers, and reduced revenues across the pork industry. The economic consequences extend beyond direct losses, affecting related sectors such as animal feed production and causing market instability due to trade restrictions and shifts in global meat supply chains.

The market valuation of ASFV vaccines highlights the production of hundreds of millions of doses annually to meet global demand, primarily due to their relatively low cost and widespread need.

The cost of African Swine Fever (ASF) vaccines varies by manufacturer and region. In Vietnam, two ASF vaccines—NAVET-ASFVAC by Navetco and AVAC ASF LIVE by AVAC Vietnam Joint Stock Company—are priced at approximately $1.50 to $1.80 per dose. The Department of Agriculture has planned to distribute 10,000 doses in the Philippines, with a cost per dose ranging from P400 to P500 (approximately $7 to $9). In the United States, where ASF vaccines are not yet commercially available, the Swine Health Information Center (SHIC) estimates the potential vaccine cost to be around $1.50 per dose. These price variations reflect differences in production costs, distribution logistics, and regional economic factors, influencing accessibility and affordability in different markets.

Additionally, the CAPEX to establish production of these vaccines is prohibitive for regions where these vaccines are direly needed, such as Africa. Therefore, there is a need to develop a cost-effective solution. The present invention presents the most cost-effective option expression of a universal ASFV vaccine in a self-replicating circular RNA that reduces the CAPEX as no biosafety issues are involved and produces a million times the number of molecules for each molecule of the RNA, making it possible to manufacture these vaccines in small facilities at almost negligible cost.

Peptide vaccines are a safer and more economical technology than traditional vaccines. The disadvantage of this technology is its poor immunogenicity. Several experiments have been performed with this vaccine to test its ability to protect animals. The published results showed that the protection reached with the peptide vaccine was lower than 50% in all the challenges tested in the field. In contrast with these results, inactivated virus vaccines (positive control) commonly reach from 90 to 100% of protection. For this reason, the vaccines routinely used as part of eradication programs and in emergencies are based on inactivated viruses.

Different serotypes of ASFV are distributed around the world. Some regions have more than one serotype and several strains, which complicate the sanitary situation and even hinder the eradication of this disease. Due to the great importance of ASFV in economic losses, it is crucial to have a vaccine that can provide cross-protection against more than one serotype and/or strain of ASFV. Thus, animals in a specific region could be protected extensively with fewer vaccination campaigns.

Vaccines based on a single virus strain often fail to protect against other strains due to a lack of cross-reactivity. Similarly, monoclonal antibodies developed against one strain do not recognize or neutralize another strain, limiting their effectiveness. To address this challenge, tailor-made peptide vaccines offer a potential solution, allowing flexibility in modifying vaccine targets. However, peptide vaccines alone have significant limitations; they are weakly immunogenic, economically unviable, and fail to elicit a strong cell-mediated immune response, essential for comprehensive immunogenic protection. The ASFV-specific cell-mediated immune response depends on the integrity and stability of the virus capsid antigen, meaning peptide vaccines alone cannot generate a robust cellular response. Moreover, research has demonstrated that cell-mediated immunity is crucial in cross-reactive protection against heterologous strains. Therefore, incorporating an inactivated ASFV component into the vaccine formulation is highly recommended, as it can enhance both humoral and cell-mediated immune responses, ultimately improving cross-protection against multiple ASFV strains.

The duration of activity of peptide vaccines, such as those used to protect against ASFV, is often limited due to the rapid clearance of peptides from the bloodstream through renal filtration and enzymatic degradation. To enhance their efficacy and reduce the frequency of booster doses, peptide vaccines can be conjugated with carriers such as albumin, transferrin, or the Fc domain of immunoglobulin. These conjugates extend the half-life of the peptide vaccine and improve its immunogenicity. Albumin conjugates leverage the natural recycling mechanism of albumin through its interaction with the neonatal Fc receptor (FcRn), significantly prolonging systemic circulation from hours to days. This can be achieved through direct fusion of peptides with albumin or by using albumin-binding peptides that attach non-covalently in vivo. Similarly, transferrin conjugates enhance delivery to antigen-presenting cells (APCs) by binding transferrin receptors (TfR) on immune cells, facilitating receptor-mediated endocytosis and efficient antigen presentation to T cells. In another strategy, Fc conjugates exploit interactions with Fc receptors on immune cells and FcRn recycling to extend the half-life of peptides and amplify immune responses via Fcγ receptor-mediated pathways. Fc fusion proteins can present peptides in multivalent formats, improving their immunogenic potential. Examples of such conjugates include peptide-albumin fusions for prolonged circulation, peptide-transferrin constructs for targeted lymphoid tissue delivery, and Fc-conjugated peptides for enhanced immune cell activation.

Additionally, these conjugates can be paired with adjuvants such as TLR agonists (e.g., CpG ODNs or Poly(I: C)), saponin-based adjuvants like QS-21, or oil-in-water emulsions such as MF59 to boost both cellular and humoral immune responses further. Challenges, however, include ensuring the stability of the conjugates during production and storage, avoiding unwanted immune responses to the carrier, and optimizing scalability for large-scale vaccine production. By using these conjugation strategies, peptide vaccines can achieve extended duration of activity, improved immune responses, and reduced frequency of administration, making them more effective and practical for widespread livestock immunization.

For the first time, the present invention introduces a platform for obtaining a wide range of vaccines that combine polynucleotides encoding viral peptides or recombinant viral peptides containing adjuvants, emulsifiers, molecular adjuvants, and carrier systems. It is proposed that the present invention could develop a universal vaccine to protect an animal against one or more viral diseases, particularly the ASFV.

### SUMMARY OF THE INVENTION

In one embodiment, the present invention provides a detailed procedure for formulating a universal vaccine capable of protecting against various serotypes and/or strains of ASF viruses.

In one embodiment, the formulations are composed of polynucleotides encoding viral peptides, polypeptides, or proteins in different types of plasmids; viral peptides or recombinant viral peptides, along with a carrier or as molecular adjuvant fused to peptides, polypeptides and/or proteins derived from viruses; adjuvants; emulsifiers, molecular adjuvants, and carrier systems.

In one embodiment, the present invention discloses a vaccine formulation capable of inducing cross-protection against different serotypes or strains of ASF viruses by (a) polynucleotides encoding peptides, polypeptides or proteins of the virus; (b) recombinant peptides, polypeptides or proteins of the virus; (c) peptides, polypeptides or proteins fused with serum proteins and (d) adjuvants to enhance the potency of the ASFV vaccine.

In one embodiment, the pharmaceutical combination comprising one or more vaccine formulations of the present invention can ensure high protection against ASFV by induction of cell-mediated and humoral components of the immunological response.

In one embodiment, the pharmaceutical combination of the present invention could be administered to domestic pigs (Sus scrofa domesticus), wild boars, warthogs, bush pigs, and giant forest hogs against ASFV infection.

In one embodiment, the pharmaceutical combination comprising one or more vaccine formulations of the present invention has the advantage of generating immunogenic cross-protection with fewer vaccination campaigns.

Extending the half-life of ASFV antigens by conjugating them with serum proteins in swine is a promising approach to improve vaccine efficacy and reduce dosing frequency. Swine albumin (porcine serum albumin, PSA) differs from human serum albumin (HSA) in amino acid sequence, structural variations, and binding affinities for specific ligands, though they share approximately 75-80% sequence identity. Both proteins exhibit similar ligand-binding sites for fatty acids, hormones, and drugs, but minor structural differences, particularly in domains I and II, may affect their binding properties. PSA's isoelectric point (pI) also differs slightly from that of HSA, influencing peptide interactions under specific conditions. Short peptides designed to bind human albumin may partially cross-bind to swine albumin, but their affinities may vary depending on sequence compatibility and the specific binding pocket involved. While some peptides with albumin-binding properties have been engineered for HSA, there is limited literature on peptides specifically targeting PSA. However, albumin-binding peptides such as GAH (GAHRMDIR) and DICLPRWGCLW have been designed to interact with HSA, and they can exhibit partial affinity toward PSA.

Albumin binding prolongs the half-life of the antigen by utilizing albumin's natural long circulation time in the bloodstream (19-21 days in humans) by leveraging the albumin recycling pathway.

### DETAILED DESCRIPTION OF THE INVENTION

The method described in this patent application illustrates the formulation process to achieve a high-quality vaccine for the ASFV virus.

In one embodiment, the present invention relates to a method to formulate a universal vaccine against one or more serotypes and/or strains of an ASFV.

In one embodiment, the present invention provides immunogenic components to formulate different vaccines to ensure cross-protection against all or different serotypes or strains of a virus, such as ASFV.

In one embodiment, the present invention provides immunogenic components to formulate different vaccines to ensure total or cross-protection against different ASFV serotypes and/or strains in combination with one or more immunogenic components ensures a high protection that comprises cellular and humoral components of the immunological response.

In one embodiment, the universal vaccine of the present invention can specifically induce one or more targeted immune responses against all or different serotypes and/or strains of an ASFV.

One of the ordinary skills in art is readily recognizing that the present invention can be designed using any combination of polynucleotides derived from ASFV.

One of the ordinary skills in art is readily recognizing that the present invention can be designed using a combination of different recombinant peptides, polypeptides, and/or proteins derived from ASFV.

Table 1. Epitopes of African Swine Virus and short peptides binding to endogenous proteins

TABLE 1
Selected UASFV Epitopes

| | | Type of | | Linker | |
|---|---|---|---|---|---|
| # | Protein Source | Epitope | Sequence | Used | Function |
| | Polyprotein p220 | CTL | TVSAIELEY | AAY | CTL Activation |
| | (CP2475L) | | (SEQUENCE NO. 1) | | |
| | Transmembrane | CTL | KTRDFFILY | AAY | CTL Activation |
| | (C62L) | | (SEQUENCE NO. 2) | | |
| | Cysteine protease | CTL | MMDFERVHY | AAY | CTL Activation |
| | (S273R) | | (SEQUENCE NO. 3) | | |
| | MGF 360-13L | CTL | KNLSIIWEY | AAY | CTL Activation |
| | | | (SEQUENCE NO. 4) | | |
| | MGF 360-18R | CTL | KAIELYWVF | AAY | CTL Activation |
| | | | (SEQUENCE NO. 5) | | |
| | Guanylyltransferase | CTL | YLYEIEIEY | AAY | CTL Activation |
| | (NP868R) | | (SEQUENCE NO. 6) | | |
| | B602L Structural | B-Cell | SKENLTPDE | KK | B-cell antibody |
| | Protein | | (SEQUENCE NO. 7) | | response |
| | CP204L (p30) | B-Cell | NECTSSFETLFE | KK | B-cell antibody |
| | Protein | | (SEQUENCE NO. 8) | | response |
| | EP153R Protein | B-Cell | SALRHLDPKKLD | KK | B-cell antibody |
| | | | (SEQUENCE NO. 9) | | response |
| | MGF-360 Protein | B-Cell | KYLDDNSLL | KK | B-cell antibody |
| | | | (SEQUENCE NO. 10) | | response |
| | E183L (p54) | B-Cell | QQWVEV | KK | B-cell antibody |
| | Protein | | (SEQUENCE NO. 11) | | response |
| | B646L (p72) | B-Cell | HKPHQSKPIL | KK | B-cell antibody |
| | Capsid | | (SEQUENCE NO. 12) | | response |
| | EP424R | HTL | ESVYFAVETIHLKQQ | GPGPG | HTL Activation |
| | | | (SEQUENCE NO. 13) | | (IL-4/IL-10) |
| | Helicase F1055L | HTL | KIGFYSSKSTAHERE | GPGPG | HTL Activation |
| | | | (SEQUENCE NO. 14) | | (IL-10) |
| | MGF505-3R | HTL | TSLFTIAISNRDLQL | GPGPG | HTL Activation |
| | | | (SEQUENCE NO. 15) | | (IL-4 and IL-10) |
| | B602L Structural | B-Cell | SKENLTPDE | KK | B-cell antibody |
| | Protein | | (SEQUENCE NO. 16) | | response |
| | CP204L (p30) | B-Cell | NECTSSFETLFE | KK | B-cell antibody |
| | Protein | | (SEQUENCE NO. 17) | | response |
| | E183L (p54) | B-Cell | QQWVEV | KK | B-cell antibody |
| | Protein | | (SEQUENCE NO. 18) | | response |
| | B646L (p72) | B-Cell | SNIKNVNKSY | KK | B-cell antibody |
| | Capsid | | (SEQUENCE NO. 19) | | response |
| | p104R | HTL | KFTVVTVKAKPARQG | GPGPG | HTL Activation |
| | | | (20) | | |
| | pF1055L | HTL | KGNYYIKNTKAGSLI | GPGPG | HTL Activation |
| | | | (SEQUENCE NO. 21) | | |
| | Minor capsid | B-Cell | GIAGRGIPLGNPHVKP | KK | B-cell antibody |
| | protein (B438L) | | (SEQUENCE NO. 22) | | response |
| | Envelope protein | B-Cell | LDAVKMDKRNIK | KK | B-cell antibody |
| | p22 (KP177R) | | (SEQUENCE NO. 23) | | response |
| | Transmembrane | B-Cell | AKLQDTKFKWKYTLDP | KK | B-cell antibody |
| | protein (C257L) | | (SEQUENCE NO. 24) | | response |
| | Major capsid | B-Cell | GRPSRRNIRFK | KK | B-cell antibody |
| | protein (B646L) | | (SEQUENCE NO. 25) | | response |

Design Elements for antigen. An N-terminal Adjuvant includes 505 ribosomal protein L7/L12 included at the beginning to enhance the immune response. The CTL (MHC-J) Epitopes are separated by AAY (Ala-Ala-Tyr) linkers to ensure proper proteasomal cleavage for CD8⁺ T-cell activation. The HTL (MHC-JJ) Epitopes are separated by GPGPG (Gly-Pro-Gly-Pro-Gly) (SEQUENCE NO. 26) linkers to enhance MHC-JJ processing and CD4⁺ T-cell activation. The B-cell Epitopes (Antibody Response) are separated by KK (Lys-Lys) linkers to provide flexibility and ensure proper folding for antibody recognition. For structural Enhancements, the EAAAK (SEQUENCE NO. 27) (Rigid Linker): is Used to separate adjuvant from epitopes and between HTL and B-cell epitopes to ensure proper folding. The GGGGS (Flexible Linker) (SEQUENCE NO. 28) is added at the end for structural stability. This Design Ensures Correct Antigen Processing as AAY allows proteasomal cleavage for CTL activation (MHC-J), the GPGPG (SEQUENCE NO. 26) spaces out HTL epitopes to avoid unwanted fusions in MHC-JJ presentation, the KK and GGGGS (SEQUENCE NO. 28) preserve B-cell epitope conformation for strong antibody production. All bringing a balance of Cellular and Humoral Immunity. It is Optimized for ASFV mRNA Vaccine Development as it can be translated directly into an ORF for mRNA-based vaccines.

>ASFV_multi_epitope_vaccine_construct_AA_sequence
(420)
(SEQUENCE NO. 29)
MSKLSSDELLDVFKEMTLLELSDFVKKFEETFEVTAAAPVSVAVAGAPAA
GEAGEAAEEQSEFDVILESAGDKKIGVIKVVREIVSGLGLKEAKDLVDGV
PKLLLEKVAKEAADDAKAKLEATGATVSVKEAAAKTVSAIELEYAAYKTR
DFFILYAAYMMDFERVHYAAYKNLSIIWEYAAYKAIELYWVFAAYYLYEI
EIEYGPGPGESVYFAVETIHLKQQGPGPGKIGFYSSKSTAHEREGPGPGT
SLFTIAISNRDLQLGPGPGKFTVVTVKAKPARQGGPGPGKGNYYIKNTKA
GSLIEAAAKSKENLTPDEKKNECTSSFETLFEKKQQWVEVKKSNIKNVNK
SYKKGIAGRGIPLGNPHVKPKKLDAVKMDKRNIKKKAKLQDTKFKWKYTL
DPKKGRPSRRNIRFKGGGGS
>ASFV_multi_epitope_vaccine_construct_CodonOptim-
ized_ORF (1266)
(SEQUENCE NO. 30)
ATGATGTCTAAGCTGTCTTCTGATGAGCTGCTGGATGTGTTTAAGGAGAT
GACTCTGCTGGAGCTGTCTGATTTTGTGAAGAAGTTTGAGGAGACTTTTG
AGGTGACTGCTGCTGCTCCTGTGTCTGTGGCTGTGGCTGGTGCTCCTGCT
GCTGGTGAGGCTGGTGAGGCTGCTGAGGAGCAGTCTGAGTTTGATGTGAT
TCTGGAGTCTGCTGGTGATAAGAAGATTGGTGTGATTAAGGTGGTGCGTG
AGATTGTGTCTGGTCTGGGTCTGAAGGAGGCTAAGGATCTGGTGGATGGT
GTGCCTAAGCTGCTGCTGGAGAAGGTGGCTAAGGAGGCTGCTGATGATGC
TAAGGCTAAGCTGGAGGCTACTGGTGCTACTGTGTCTGTGAAGGAGGCTG
CTGCTAAGACTGTGTCTGCTATTGAGCTGGAGTATGCTGCTTATAAGACT
CGTGATTTTTTTATTCTGTATGCTGCTTATATGATGGATTTTGAGCGTGT
GCATTATGCTGCTTATAAGAACCTGTCTATTATTTGGGAGTATGCTGCTT
ATAAGGCTATTGAGCTGTATTGGGTGTTTGCTGCTTATTATCTGTATGAG
ATTGAGATTGAGTATGGTCCTGGTCCTGGTGAGTCTGTGTATTTTGCTGT
GGAGACTATTCATCTGAAGCAGCAGGGTCCTGGTCCTGGTAAGATTGGTT
TTTATTCTTCTAAGTCTACTGCTCATGAGCGTGAGGGTCCTGGTCCTGGT
ACTTCTCTGTTTACTATTGCTATTTCTAACCGTGATCTGCAGCTGGGTCC
TGGTCCTGGTAAGTTTACTGTGGTGACTGTGAAGGCTAAGCCTGCTCGTC
AGGGTGGTCCTGGTCCTGGTAAGGGTAACTATTATATTAAGAACACTAAG
GCTGGTTCTCTGATTGAGGCTGCTGCTAAGTCTAAGGAGAACCTGACTCC
TGATGAGAAGAAGAACGAGTGTACTTCTTCTTTTGAGACTCTGTTTGAGA
AGAAGCAGCAGTGGGTGGAGGTGAAGAAGTCTAACATTAAGAACGTGAAC
AAGTCTTATAAGAAGGGTATTGCTGGTCGTGGTATTCCTCTGGGTAACCC
TCATGTGAAGCCTAAGAAGCTGGATGCTGTGAAGATGGATAAGCGTAACA
TTAAGAAGAAGGCTAAGCTGCAGGATACTAAGTTTAAGTGGAAGTATACT
CTGGATCCTAAGAAGGGTCGTCCTTCTCGTCGTAACATTCGTTTTAAGGG
TGGTGGTGGTTCTTGA

Short peptides can be additionally bound to antigens to promote their half-life (Table 2)

TABLE 2
Short peptides to bind albumin, Fc and transferrin

| | Target Endogenous | |
|---|---|---|
| Peptide Sequence | Protein | Binding Function |
| GAHRMDIR | Swine Albumin | Albumin-binding peptide for |
| (SEQUENCE NO. 31) | | extended half-life |
| DICLPRWGCLW | Swine Albumin | Albumin-binding peptide for |
| (SEQUENCE NO. 32) | | enhanced stability |
| ADQVSDQTLNAVVHVFK | Swine Albumin | Fusion peptide for vaccine |
| (SEQUENCE NO. 33) | | stabilization |
| NQDKQLAALNDVGAKAA | Swine Albumin | Fusion protein expression, |
| (SEQUENCE NO. 34) | | stabilization |
| FYWHCLDE | Swine FcRn | Enhancing FcRn recycling to |
| (SEQUENCE NO. 35) | (Albumin Recycling) | extend half-life |
| Ac- | Swine Albumin | Synthetic peptide designed for in |
| RLIEDICLPRWGCLWEDD-NH2 | | vivo albumin binding for in vitro |
| (SEQUENCE NO. 36) | | conjugation |
| QYDDAVRR | Swine Transferrin | Enhances receptor-mediated uptake |
| (SEQUENCE NO. 37) | | via TfR |
| TNLLSAQ | Swine Transferrin | Improves transferrin binding and |
| (SEQUENCE NO. 38) | | delivery |
| WQVDGRG | Swine Transferrin | Facilitates antigen attachment to |
| (SEQUENCE NO. 39) | | transferrin |
| FNKYIEH | Swine Transferrin | Supports prolonged antigen circulation |
| (SEQUENCE NO. 40) | | |
| LGGHAEK | Swine Transferrin | Enhances vaccine targeting through |
| (SEQUENCE NO. 41) | | transferrin transport |

### Fusing Carrier Proteins In Vitro

The process involves carefully selecting a conjugation strategy, protein engineering, purification, and ligation to fuse a sequence of conjugated epitopes of ASFV with albumin or via a conceivable peptide bond in vitro. Two primary methods are commonly employed: Sortase-Mediated Ligation (SML) and chemical ligation. In the SML approach, Sortase A, a transpeptidase, recognizes a specific sequence motif (e.g., LPXTG) engineered to target protein and catalyzes the formation of a peptide bond. In chemical ligation, reactive groups, such as cysteine residues, are introduced at specific sites on transferrin and the target protein to enable covalent bonding through thiol-based or other chemistries, such as carbodiimide-mediated amide bond formation or click chemistry.

### Genetic Fusion

The genetic fusion begins with the in-silico design of the DNA sequence. The open reading frame (ORF) for the protein of interest is joined with the ORF of the ligand (albumin or a short peptide) through a DNA sequence encoding the non-cleavable peptide linker. Codon optimization is performed to ensure efficient expression in the chosen host system, whether bacterial, yeast, or mammalian cells. For albumin and transferrin, mammalian expression systems are often preferred due to the need for proper folding and post-translational modifications.

The current vaccine already aligns with the principle of using a protein-based adjuvant (L7/L12) to enhance immunogenicity. However, additional adjuvants (e.g., Flagellin, PADRE, GM-CSF, or Fc fusion) could be incorporated to further boost the innate and adaptive immune response.

The construct is inserted into an expression vector with suitable regulatory elements, such as a strong promoter, signal peptide for secretion, and a polyadenylation signal for eukaryotic systems. After transformation into the host cells, the fusion protein is expressed as a single polypeptide chain with the ligand and protein connected by the non-cleavable peptide linker. The expressed fusion protein is then purified using affinity chromatography, exploiting tags like His-tags or natural binding affinities (e.g., Fc binding to Protein A/G or albumin's natural affinity to specific resins).

### RNA

Linear mRNA Structure: Linear mRNA vaccines are single-stranded RNA molecules engineered to mimic the structure of natural mRNA.

Basic Structure (without Self-Replication Components)

5′ Cap: A chemically modified guanosine molecule at the 5′ end, enhancing stability and translation efficiency. Untranslated Regions (UTRs): Sequences flanking the coding region that improve RNA stability and translation. Open Reading Frame (ORF): The coding sequence for the antigenic protein. This is the central part that gets translated into the protein of interest. Poly-A Tail: A string of adenosines at the 3′ end that increases stability and prolongs the mRNA's lifespan in the cytoplasm.

### Enhanced Structure (With Self-Replication Components):

Replicase Genes: Sequences encoding an RNA-dependent RNA polymerase (e.g., derived from alphaviruses like the Semliki Forest virus) are added. These enzymes amplify the mRNA within the cell, allowing for prolonged and enhanced protein production. Subgenomic Promoter: Facilitates transcription of the ORF while keeping replicase separate.

Circular RNA (circRNA) Structure

Circular RNA vaccines are covalently closed-loop RNA molecules. They offer increased stability and resistance to exonucleases compared to linear mRNA.

### Basic Structure (Without Self-Replication Components):

Closed Circular Form: The RNA ends are covalently linked, creating a stable loop.

Internal Ribosome Entry Site (IRES): A sequence enabling ribosome binding and translation initiation, bypassing the need for a 5′ cap. Coding Region: The antigenic protein-encoding sequence is similar to linear mRNA. UTRs: Help in translation regulation and RNA stability.

### Enhanced Structure (With Self-Replication Components):

Replicase Genes: Like linear mRNA, replicase genes can be included to enable self-replication. Translation Enhancers: Additional elements to boost protein expression, given the unique topology of circRNA.

Delivering a genetically fused protein with albumin using linear mRNA or circular RNA (replicating or non-replicating) involves carefully designing RNA constructs that encode the fusion protein, including the ligand, target protein, and a non-cleavable peptide linker. The RNA sequence is engineered to include essential elements for efficient expression. For linear mRNA, these include a 5′ cap for translation initiation, a 5′ untranslated region (UTR) optimized for translational efficiency, an open reading frame (ORF) encoding the fusion protein, and a 3′ UTR and poly(A) tail to ensure stability and translation. For circular RNA, circularization elements such as ribozyme sequences or exonuclease-resistant motifs are included to enable covalent circularization. Codon optimization enhances expression in the host system, such as human cells, while minimizing secondary structures that could impede translation.

An adjuvant is generally unnecessary for an mRNA vaccine encapsulated in lipid nanoparticles (LNPs). LNPs themselves serve as both delivery systems and inherent adjuvants, primarily due to their ability to Enhance Cellular Uptake. LNPs facilitate the delivery of mRNA into cells, enabling the translation of the mRNA into the target antigen. Induce Innate Immune Response: The mRNA within the LNP can stimulate innate immune pathways, such as Toll-like receptors (TLRs) 3, 7, and 8, and other RNA sensors (e.g., RIG-I, MDA5). These innate responses promote the activation of antigen-presenting cells, such as dendritic cells, which are crucial for initiating adaptive immunity. LNPs protect mRNA from enzymatic degradation and facilitate efficient delivery to the cytoplasm. For most applications, an mRNA vaccine with LNPs does not require a separate adjuvant because LNPs already perform multiple functions, including enhancing immunogenicity. However, specific vaccine designs or target populations might occasionally benefit from adjuvant inclusion to tailor or boost immune responses.

mRNA vaccines inherently stimulate TLRs (e.g., TLR3, TLR7, TLR8) due to the presence of single-stranded RNA, inducing innate immunity.

Adding a TLR9-based adjuvant can provide synergistic immune activation, mainly to boost specific immune pathways. CpG Oligonucleotides as TLR9 Agonists: CpG-based adjuvants are compatible with mRNA vaccines and have been used in various vaccine platforms. They can enhance antibody titers and quality, improve the durability of the immune response, and provide a dose-sparing effect, which is crucial for large-scale vaccination efforts. An example of a CpG-based TLR9 agonist is CpG 1018, a synthetic oligodeoxynucleotide (ODN) containing unmethylated CpG motifs. CpG 1018 is widely used as an adjuvant in human and veterinary vaccines. It is one of the most prominent examples of a CpG-based TLR9 agonist approved and successfully used in vaccines. Its demonstrated ability to enhance both humoral and cellular immunity makes it a viable option for designing new vaccines, including ASFV.

Depending on the vaccine's design and immunogenic requirements, flagellin-based adjuvants could be a promising choice for an mRNA vaccine targeting ASFV.

The construction of non-replicating linear mRNA begins with in vitro transcription (IVT) using a DNA template containing the RNA sequence under a T7 or SP6 promoter. The resulting RNA is capped using methods (e.g., CleanCap) and polyadenylated during transcription or enzymatically post-transcription. The RNA sequence includes viral replicon elements for replicating linear mRNA, such as the non-structural proteins (e.g., nsPl-nsP4) from alphaviruses, enabling intracellular RNA replication. The ORF encoding the fusion protein is placed downstream of a subgenomic promoter within the replicon sequence. The RNA is purified to remove impurities and unwanted by-products, ensuring high-quality RNA for delivery.

Circular RNA is constructed by designing a linear RNA sequence with flanking ribozyme sequences or self-splicing introns, which mediate intramolecular ligation and produce a covalently closed RNA molecule. Alternatively, enzymatic circularization can be performed using RNA ligases. Replicating circular RNA includes additional viral elements that allow intracellular replication, leveraging the stability of the circular structure and prolonging protein expression. Non-replicating and replicating circular RNA are purified to eliminate any remaining linear RNA, ensuring homogeneity of the final product.

The RNA constructs, whether linear or circular, are delivered into cells using lipid nanoparticles (LNPs), which protect the RNA from degradation and facilitate cellular uptake. Alternatively, electroporation or polymeric nanoparticles may be employed depending on the application. Once inside the cells, the host's machinery translates the RNA to produce the genetically fused protein. Linear mRNA offers a more straightforward construction process and is ideal for transient expression while replicating mRNA provides extended protein expression with lower RNA doses. Circular RNA, on the other hand, is inherently more stable than linear NA due to its resistance to exonucleases, and its replicating variant combines this stability with sustained expression through intracellular replication.

For all RNA types, the ORF encodes the genetically fused protein, starting with the target protein, followed by a non-cleavable peptide linker (e.g., Gly-Ser-Gly-Ser-Gly), and ending with the ligand, such as albumin, transferrin, or an Fc fragment. The resulting fusion protein benefits from the ligand's properties: albumin extends the protein's half-life, transferrin facilitates receptor-mediated delivery, and the Fc fragment enhances therapeutic properties and immune interactions. These RNA platforms are versatile, scalable, and suitable for a wide range of therapeutic and research applications, offering distinct advantages in stability, duration of expression, and ease of design.

Linear peptides can be expressed using RNA by designing a sequence that encodes the peptide of interest, enabling host cells to synthesize the desired linear peptide efficiently. Unlike dendrimeric peptides, linear peptides have a straightforward structure without branching, making them easier to design and express. The RNA construct is designed to include essential regulatory elements for effective translation, such as a 5′ cap for initiating translation, a 5′ untranslated region (UTR) optimized for efficient ribosome binding, the open reading frame (ORF) encoding the peptide sequence, a 3′ UTR for stability and translational regulation, and a poly(A) tail to enhance mRNA stability and prolong translation. If the peptide requires secretion, a signal peptide is added to the sequence to direct the peptide to the endoplasmic reticulum (ER) for proper processing and secretion.

The coding sequence (ORF) encodes explicitly the linear peptide, which may include additional elements like flexible linkers (e.g., Gly-Ser-Gly) to enhance stability or allow for modular design if multiple epitopes or functional domains are included. Codon optimization ensures high translational efficiency in the target host system, mammalian or another cell type. RNA is synthesized in vitro using a DNA template under a T7 or SP6 promoter, producing high-quality RNA. This RNA may be linear mRNA, replicating RNA, or circular RNA, depending on the desired duration and yield of peptide expression. Non-replicating linear RNA provides short-term expression, while replicating RNA includes viral replicons that allow intracellular amplification, resulting in sustained peptide production. Circular RNA offers enhanced stability due to resistance to exonucleases and combines this stability with prolonged antigen expression when replicating elements are added.

The RNA is delivered into host cells using lipid nanoparticles (LNPs), which protect the RNA from degradation and ensure efficient cellular uptake. Once inside the cytoplasm, the host's ribosomes translate the RNA into the linear peptide. If a signal peptide is included, the peptide is routed through the ER for proper folding, processing, and secretion into the extracellular space. The peptide can act as an immunogen or therapeutic agent, depending on its intended application. Linear peptides encoded by RNA are suitable for vaccine development, as they can present antigenic epitopes from pathogens like ASFV. For example, a linear RNA encoding peptides derived from the VP1 protein of ASFV, including the hypervariable GH loop region, can elicit a robust immune response, particularly when paired with suitable adjuvants.

Linear peptides expressed via RNA delivery are advantageous due to their simplicity, scalability, and ease of production. They can be tailored to include immunodominant epitopes to enhance immune recognition and are more stable when expressed using advanced RNA delivery methods like LNPs. However, challenges include optimizing RNA stability, translation efficiency, and immunogenicity while avoiding degradation and unwanted inflammatory responses. This approach provides a versatile, cost-effective platform for producing linear peptides for vaccines, therapeutics, and research applications. For instance, combining RNA-delivered linear peptides with adjuvants like CpG ODNs, QS-21, or MPL can significantly enhance the immunogenicity of peptide-based vaccines.

Circular RNA (circRNA) is a versatile platform for delivering linear peptides by encoding the desired peptide sequence within a covalently closed RNA molecule. The circular structure resists exonucleases, making circRNA significantly more stable than linear RNA, and enables prolonged expression of the encoded peptide in the host cells. This inherent stability and ability to sustain peptide production make circRNA an ideal candidate for vaccine applications, therapeutics, and diagnostics. Expressing linear peptides using circRNA involves careful design of the RNA construct to ensure efficient translation and functionality of the peptide.

The circRNA construct includes an open reading frame (ORF) encoding the linear peptide sequence, which may feature immunogenic epitopes, therapeutic sequences, or functional domains tailored to the intended application. The ORF can also incorporate design elements such as signal peptides to direct the linear peptide to the secretory pathway for extracellular release or flexible linkers (e.g., Gly-Ser-Gly) for enhanced stability and modular design. Regulatory elements are integrated to facilitate translation, including internal ribosome entry sites (IRES) or translation-enhancing sequences like optimized UTRs, which allow cap-independent translation in eukaryotic cells. Codon optimization ensures high translational efficiency in the host system, minimizing secondary structures that could impede ribosomal binding and elongation.

To generate circRNA, the RNA is first transcribed as a linear precursor from a DNA template containing self-splicing ribozyme sequences or engineered splice sites at the flanking regions of the ORF. These elements facilitate intramolecular ligation or splicing, creating a covalently closed circular RNA molecule. Alternatively, enzymatic circularization methods, such as RNA ligase-mediated reactions, can generate high-purity circRNA. The circularization process eliminates free RNA ends, protecting the circRNA from exonuclease degradation and providing an extended half-life compared to linear RNA.

The circRNA is delivered into host cells using advanced delivery systems like lipid nanoparticles (LNPs), which protect the RNA from degradation and enhance cellular uptake. Once inside the cytoplasm, the host's translational machinery recognizes the IRES or other translation-initiation elements, allowing efficient production of the linear peptide encoded within the circRNA. If a signal peptide is included in the ORF, the expressed peptide is directed to the endoplasmic reticulum (ER) for proper folding, processing, and secretion into the extracellular space. This process ensures that the peptide functions effectively as an immunogen or therapeutic agent, depending on its intended application.

The use of circRNA for linear peptide delivery offers several advantages, including enhanced stability, prolonged peptide expression, and the ability to achieve sustained immune stimulation or therapeutic effects. For vaccine development, circRNA can encode linear peptides derived from highly immunogenic regions of pathogens, such as the VP1 GH loop of ASFV. This approach enables robust immune responses while reducing the need for frequent dosing. CircRNA is also inherently more stable during storage and transport, making it well-suited for scalable manufacturing and global distribution.

CircRNA delivery faces challenges despite its benefits, including optimizing translation efficiency and ensuring uniform circularization. Advances in RNA engineering, such as improved IRES sequences and high-yield circularization methods, address these limitations and enhance the effectiveness of circRNA-based platforms. Additionally, circRNA can be combined with immunostimulatory adjuvants like CpG ODNs, MPL, or QS-21 to amplify immune responses in vaccine applications. Overall, circular RNA provides a highly stable and efficient platform for delivering linear peptides, offering significant potential in biomedical research and clinical applications.

Self-replicating linear and circular RNA constructs offer an innovative approach for delivering peptides, leveraging their ability to amplify intracellular RNA levels, leading to sustained peptide production over extended periods. These constructs incorporate self-replicating design elements, typically derived from viral replicons, which enable autonomous amplification within the host cells. This strategy significantly enhances the yield of the encoded peptides, reducing the RNA dosage required for adequate therapeutic or immunogenic responses. Both linear and circular self-replicating RNA designs utilize similar principles but differ in structural characteristics and stability.

In self-replicating linear RNA, the construct includes essential replication elements such as non-structural protein (nsP) sequences from RNA viruses like alphaviruses or flaviviruses. These nsP sequences encode RNA-dependent RNA polymerase and accessory proteins that drive replication of the RNA template within the cytoplasm. The RNA construct is designed with a 5′ cap to initiate translation, followed by untranslated regions (UTRs) optimized for stability and replication efficiency. A subgenomic promoter is included downstream of the nsP sequences to drive the expression of the open reading frame (ORF) encoding the target peptide. The ORF can include functional elements such as signal peptides for secretion, flexible linkers, or fusion domains to enhance the stability and activity of the peptide. The 3′ UTR and poly(A) tail are engineered for efficient replication and translational control. Upon delivery into host cells using lipid nanoparticles (LNPs), the self-replicating RNA initiates replication and translation, producing high peptide levels. The transient process provides sustained expression over several days, enabling robust immunogenic or therapeutic effects.

The construct builds on the inherent stability of circular RNA for self-replicating circular RNA while incorporating replicative capabilities. The circular RNA is engineered with viral replication elements, such as replicon sequences from alphaviruses or flaviviruses, that enable intracellular RNA amplification. A ribozyme or enzymatic ligation generates a covalently closed circular structure, eliminating free RNA ends and protecting the molecule from exonucleases. The ORF encoding the peptide is placed downstream of a subgenomic promoter within the replicon, allowing efficient translation of the peptide once replication is initiated. Translation initiation in circular RNA constructs typically relies on internal ribosome entry sites (IRES) or other cap-independent elements, as circular RNA lacks a 5′ cap. The combination of replication-driven amplification and the stability of the circular RNA structure results in prolonged expression of the target peptide, often lasting weeks.

In both linear and circular self-replicating RNA, the target peptide can be designed to include immunogenic epitopes, therapeutic sequences, or diagnostic markers. For example, peptides derived from the VP1 GH loop of ASFV can be encoded in the RNA constructs, providing a strong immunogenic response in vaccine applications. The high yield of peptide production from self-replicating RNA reduces the need for frequent dosing, making it particularly advantageous for scalable vaccine development. Additionally, the constructs can be combined with immunostimulatory adjuvants, such as CpG ODNs, MPL, or QS-21, to enhance the immune response further.

Whether linear or circular, self-replicating RNA systems offer distinct advantages, including high expression levels, prolonged peptide production, and reduced RNA dosages. Linear RNA systems are more straightforward to design and manufacture, while circular RNA systems provide enhanced stability and resistance to degradation. Both systems face challenges, such as ensuring efficient delivery, minimizing off-target effects, and optimizing replication and translation efficiency. Advances in RNA engineering, delivery technologies like LNPs, and integrating stabilizing elements continue to improve the feasibility and effectiveness of self-replicating RNA platforms for peptide delivery. This approach holds immense potential for applications in vaccines, therapeutics, and beyond.

The comparative yield of protein expression from non-replicating linear mRNA, replicating linear mRNA, non-replicating circular RNA, and replicating circular RNA depends on factors such as stability, translation efficiency, and the duration of protein expression. Below is a general comparison of their expected relative yields based on these properties:
a) The comparative yield of protein expression from non-replicating linear mRNA, replicating linear mRNA, non-replicating circular RNA, and replicating circular RNA depends on factors such as stability, translation efficiency, and the duration of protein expression. Table 6 lists a general comparison of their expected relative yields based on these properties.
b) The relative quantitative yield of protein expression from the four RNA types can be approximated based on experimental data and known differences in stability, replication, and translation efficiency.

In one embodiment, the present invention can be designed with any peptide polypeptide or protein as carriers fused to various epitopes derived from ASFV.

### Adjuvants

The present invention can be designed using different adjuvants, emulsifiers, molecular adjuvants, and carrier systems. In one embodiment, the formulation of the present invention includes, but not limited to, TLR Agonists: CPG (TLR9), Poly I:C (TLR3), MPL (TLR4); Saponin-Based Adjuvants: QS-21 (for protein vaccines) Liposomes/LNPs: Required for mRNA delivery; Oil-in-Water Emulsions: Montanide ISA series, Emulsigen, ISCOMS (for protein, not mRNA). Aluminum-based adjuvants and Induce Th2 responses are not recommended, whereas ASFV needs Th1-biased immunity and Freund's adjuvant as they are roo reactogenic for veterinary use.

In one embodiment, the universal vaccine of the present invention could be administered by syringe injection, needle-free injection, microneedle patch, and delivery. The pharmaceutical combination can be administered by different routes, such as oral, intramuscular (IM), subcutaneous (SC), intradermal (ID), and intranasal spray (INS).

In one embodiment, the vaccine formulations of the present invention utilize proteins as carriers of foreign peptides, polypeptides, and/or proteins. In one embodiment, the albumin, transferrin, or Fc is used as a carrier or molecular adjuvant to redirect the immune response towards a specific strain or serotype. In some embodiments, the N-amino end of the carriage protein is fused to a foreign peptide, polypeptides, and/or proteins.

In various embodiments, the universal vaccine can be formulated using linear peptide epitopes in tandem. In another embodiment, a combination of T and B epitopes can be used

In one embodiment, the universal vaccine formulation comprises one or more carrier proteins, chimeric proteins carrying different peptides, polypeptides, and/or proteins derived from different ASFV types to give protection against various kinds of ASFV.

In another embodiment, different peptides, polypeptides, and/or proteins from ASFV can be fused to the carrier protein-protein to protect against various types of ASFV.

In certain embodiments, one or more peptides, polypeptides, and/or proteins from ASFV fused to the carrier proteins protein derived from B epitopes and/or T epitopes of ASFV.

In one embodiment, the criteria for choosing different peptides, polypeptides, and/or proteins from ASFV to be fused with carrier proteins depend on the type of ASFV one needs protection against. This invention can design vaccines against one specific strain, against different strains of the same serotype, or other serotypes. For protection against a particular strain, a combination of peptides derived from the B and T epitope of that strain is recommended to reach a high protection level. For protection against more than one strain, it is necessary to combine peptides derived from epitopes of different strains, preferably peptides derived from both B epitope and T epitope of ASFV. For protection against more than one serotype, it is necessary to combine peptides derived from epitopes of different serotypes, preferably peptides derived from both B epitope and T epitope.

In one embodiment, examples of peptides with ASFV epitopes include but are not limited to, the peptides described in Table 1. One would recognize that the present invention is not limited to the following peptides: it encompasses the following peptides and their variants, homologous sequences, and/or functional analogs.

In one embodiment, the present invention is suitable to qualify as an emergency vaccine under the OIE (World Organisation for Animal Health) protocol to be used against outbreaks of emerging ASFV strains. This qualification is achieved because this vaccine provides animals with sufficient protection against ASFV infection after a single administration.

In one embodiment, the present invention is suitable to generate antigen banks that could be used in an emergency to formulate an ASFV universal vaccine.

In another embodiment, the vaccine formulations can be administered in multiple doses.

In one embodiment, the present invention provides a vaccine formulation capable of inducing cross-protection against different serotypes and/or strains of ASFV, comprising encoding ASFV peptides, polypeptides or proteins in various types of plasmids; b) recombinant ASFV peptides, polypeptides or proteins; c) peptides, polypeptides or proteins used as a carrier or as molecular adjuvant fused to peptides, polypeptides and/or proteins derived from ASFV; d) adjuvants; emulsifiers, molecular adjuvants and carrier systems.

In one embodiment, the above vaccine formulation can induce protective immunity against all strains of a given serotype of ASFV. In another embodiment, the vaccine formulation can induce protective immunity against all strains of one or more serotypes. In another embodiment, the vaccine formulation can induce protective immunity against all strains of all serotypes of ASFV.

In one embodiment, the vaccine formulation comprises peptides, polypeptides, and proteins used as carriers and/or molecular adjuvants that are fused to entire or, partial, or variant amino acid sequences of one or more ASFV peptides, polypeptides, and proteins.

In another embodiment, the carrier proteins are fused to peptides, polypeptides, and proteins with any peptide or polypeptide linker. In one embodiment, the carrier proteins are fused to one or more ASFV peptides, polypeptides, and proteins from the same ASFV strain or serotype. In another embodiment, the carrier proteins are fused to one or more ASFV peptides, polypeptides, and proteins from different ASFV strains and/or serotypes. In another embodiment, the carrier proteins or their variants are not fused to ASFV peptides, polypeptides, and proteins. In another embodiment, variants of carrier proteins are carrier proteins with point mutations that improve the degree of stability.

In one embodiment, the vaccine formulation is an emulsion, such as a water-in-oil emulsion (W/O), an oil-in-water (O/W) emulsion, or a water-in-oil-in water emulsion (W/O/W), or an oil-in-water-in oil (O/W/O) emulsion. In another embodiment, the vaccine formulation comprises a mix of an emulsion and one or more additional adjuvants.

The vaccine formulation comprises a lipid nanoparticle (LNP) in one embodiment.

In one embodiment, the carrier systems can be liposomes, microspheres, nanoparticles, micellar systems, or immune stimulating complexes (ISCOMs).

The present invention also provides a method of vaccinating a host susceptible to ASFV infection, comprising administrating the vaccine formulation described above to the host to induce an immune response.

In one embodiment, the pharmaceutical combination can be administered simultaneously, along with the exact body location of the host. In another embodiment, the pharmaceutical combination can be administered at different times and the precise body location of the host. In another embodiment, the pharmaceutical combination can be administered simultaneously and at various body locations of the host. In another embodiment, the pharmaceutical combination can be administered at different times and other body locations of the host.

1. A universal multi-epitope vaccine formulation capable of inducing cross-protection against multiple genotypes (I-XXIV) and serotypes (1-8) of African Swine Fever Virus (ASFV), comprising:
a. a plurality of peptide epitopes selected from ASFV structural proteins (p72, p54, p30, CD2v) and non-structural proteins (MGF family, DNA replication enzymes, and immune evasion factors) to induce cellular (CD8⁺ and CD4⁺ T-cell) and humoral (antibody-mediated) immunity.
b. optimized linker sequences, wherein Cytotoxic T-lymphocyte (CTL) epitopes are connected via AAY (Ala-Ala-Tyr) linkers for MHC-I presentation, Helper T-lymphocyte (HTL) epitopes are connected via GPGPG (Gly-Pro-Gly-Pro-Gly) (SEQUENCE NO. 27), linkers for MHC-II processing, and B-cell epitopes are connected via KK (Lys-Lys) or GGGGS (Gly-Gly-Gly-Gly-Ser) (SEQUENCE NO. 29) linkers to enhance antibody recognition.
c. an adjuvant selected from the group consisting of TLR agonists (CpG ODN, Poly I:C, MPL), Saponin-based adjuvants (e.g., QS-21), and Oil-in-water emulsions (e.g., Montanide ISA) and Lipid nanoparticles (LNPs) for mRNA encapsulation.
d. a carrier system to enhance vaccine half-life and antigen uptake, wherein the antigen is fused to a serum protein or peptide selected from Albumin, Transferrin, or Fc fusion peptides.
e. a formulation produced by either recombinant expression of antigenic proteins or mRNA-based technology, including linear mRNA, self-replicating RNA, or circular RNA.
f. a pharmaceutical composition suitable for administration via Intramuscular (IM), subcutaneous (SC), intradermal (ID), oral, or intranasal spray delivery.”

2. The universal multi-epitope vaccine formulation of claim 1, wherein, the peptide epitope comprise TVSAIELEY (SEQUENCE NO. 1), KTRDFFILY (SEQUENCE NO. 2), MMDFERVHY (SEQUENCE NO. 3), KNLSIIWEY (SEQUENCE NO. 4), KAIELYWVF (SEQUENCE NO. 5), YLYEIEIEY (SEQUENCE NO. 6), SKENLTPDE (SEQUENCE NO. 7), NECTSSFETLFE (SEQUENCE NO. 8), SALRHLDPKKLD (SEQUENCE NO. 9), KYLDDNSLL (SEQUENCE NO. 10), QQWVEV (SEQUENCE NO. 11), HKPHQSKPIL (SEQUENCE NO. 12), ESVYFAVETIHLKQQ (SEQUENCE NO. 13), KIGFYSSKSTAHERE (SEQUENCE NO. 14), TSLFTIAISNRDLQL (SEQUENCE NO. 15), SKENLTPDE (SEQUENCE NO. 16), NECTSSFETLFE (SEQUENCE NO. 17), QQWVEV (SEQUENCE NO. 18), SNIKNVNKSY (SEQUENCE NO. 19), KFTVVTVKAKPARQG (20), KGNYYIKNTKAGSLI (SEQUENCE NO. 21), GIAGRGIPLGNPHVKP (SEQUENCE NO. 22), LDAVKMDKRNIK (SEQUENCE NO. 23), AKLQDTKFKWKYTLDP (SEQUENCE NO. 24), and GRPSRRNIRFK (SEQUENCE NO. 25) or a combination thereof.

3. The universal multi-epitope vaccine formulation of claim 1, wherein the antigen is a 420 amino acid construct comprising
(SEQUENCE NO. 29)
MSKLSSDELLDVFKEMTLLELSDFVKKFEETFEVTAAAPVSVAVAGAPAA
GEAGEAAEEQSEFDVILESAGDKKIGVIKVVREIVSGLGLKEAKDLVDGV
PKLLLEKVAKEAADDAKAKLEATGATVSVKEAAAKTVSAIELEYAAYKTR
DFFILYAAYMMDFERVHYAAYKNLSIIWEYAAYKAIELYWVFAAYYLYEI
EIEYGPGPGESVYFAVETIHLKQQGPGPGKIGFYSSKSTAHEREGPGPGT
SLFTIAISNRDLQLGPGPGKFTVVTVKAKPARQGGPGPGKGNYYIKNTKA
GSLIEAAAKSKENLTPDEKKNECTSSFETLFEKKQQWVEVKKSNIKNVNK
SYKKGIAGRGIPLGNPHVKPKKLDAVKMDKRNIKKKAKLQDTKFKWKYTL
DPKKGRPSRRNIRFKGGGGS.

4. The universal multi-epitope vaccine formulation of claim 1, wherein an open reading frame for mRNA production comprises of 1266 units as
(SEQUENCE NO. 30)
ATGATGTCTAAGCTGTCTTCTGATGAGCTGCTGGATGTGTTTAAGGAGAT
GACTCTGCTGGAGCTGTCTGATTTTGTGAAGAAGTTTGAGGAGACTTTTG
AGGTGACTGCTGCTGCTCCTGTGTCTGTGGCTGTGGCTGGTGCTCCTGCT
GCTGGTGAGGCTGGTGAGGCTGCTGAGGAGCAGTCTGAGTTTGATGTGAT
TCTGGAGTCTGCTGGTGATAAGAAGATTGGTGTGATTAAGGTGGTGCGTG
AGATTGTGTCTGGTCTGGGTCTGAAGGAGGCTAAGGATCTGGTGGATGGT
GTGCCTAAGCTGCTGCTGGAGAAGGTGGCTAAGGAGGCTGCTGATGATGC
TAAGGCTAAGCTGGAGGCTACTGGTGCTACTGTGTCTGTGAAGGAGGCTG
CTGCTAAGACTGTGTCTGCTATTGAGCTGGAGTATGCTGCTTATAAGACT
CGTGATTTTTTTATTCTGTATGCTGCTTATATGATGGATTTTGAGCGTGT
GCATTATGCTGCTTATAAGAACCTGTCTATTATTTGGGAGTATGCTGCTT
ATAAGGCTATTGAGCTGTATTGGGTGTTTGCTGCTTATTATCTGTATGAG
ATTGAGATTGAGTATGGTCCTGGTCCTGGTGAGTCTGTGTATTTTGCTGT
GGAGACTATTCATCTGAAGCAGCAGGGTCCTGGTCCTGGTAAGATTGGTT
TTTATTCTTCTAAGTCTACTGCTCATGAGCGTGAGGGTCCTGGTCCTGGT
ACTTCTCTGTTTACTATTGCTATTTCTAACCGTGATCTGCAGCTGGGTCC
TGGTCCTGGTAAGTTTACTGTGGTGACTGTGAAGGCTAAGCCTGCTCGTC
AGGGTGGTCCTGGTCCTGGTAAGGGTAACTATTATATTAAGAACACTAAG
GCTGGTTCTCTGATTGAGGCTGCTGCTAAGTCTAAGGAGAACCTGACTCC
TGATGAGAAGAAGAACGAGTGTACTTCTTCTTTTGAGACTCTGTTTGAGA
AGAAGCAGCAGTGGGTGGAGGTGAAGAAGTCTAACATTAAGAACGTGAAC
AAGTCTTATAAGAAGGGTATTGCTGGTCGTGGTATTCCTCTGGGTAACCC
TCATGTGAAGCCTAAGAAGCTGGATGCTGTGAAGATGGATAAGCGTAACA
TTAAGAAGAAGGCTAAGCTGCAGGATACTAAGTTTAAGTGGAAGTATACT
CTGGATCCTAAGAAGGGTCGTCCTTCTCGTCGTAACATTCGTTTTAAGGG
TGGTGGTGGTTCTTGA.

5. The universal multi-epitope vaccine formulation of claim 1, wherein the peptide epitopes are additionally fused with short peptides capable of binding with endogenous serum proteins comprising albumin, Fc, and transferrin, or a combination thereof.

6. The universal multi-epitope vaccine formulation of claim 5, wherein the short peptide comprise, GAHRMDIR (SEQUENCE NO. 31), DICLPRWGCLW (SEQUENCE NO. 32), ADQVSDQTLNAVVHVFK (SEQUENCE NO. 33), NQDKQLAALNDVGAKAA (SEQUENCE NO. 34), FYWHCLDE (SEQUENCE NO. 35), RLIEDICLPRWGCLWEDD (SEQUENCE NO. 36), QYDDAVRR (SEQUENCE NO. 37), TNLLSAQ (SEQUENCE NO. 38), WQVDGRG (SEQUENCE NO. 39), FNKYIEH (SEQUENCE NO. 40), and LGGHAEK (SEQUENCE NO. 41), or a combination thereof.

7. The universal multi-epitope vaccine formulation of claim 1, wherein the serum proteins are fused in vitro by a chemical reaction, expressed as a combined protein using recombinant technology or delivered using linear mRNA, circular RNA, both with and without self-replication.

8. A method of vaccinating a host susceptible to ASFV infection, comprising administrating one or more of the vaccine formulations of claim 1 to induce an immune response in a host comprising domestic pigs (Sus scrofa domesticus), wild boars, warthogs, bush pigs, and giant forest hogs.

9. The method of claim 8, wherein the induced immune response comprises cross-protective neutralizing antibodies against two or more serotypes or strains of ASFV.
